(19) United States
(12) Patent Application Publication
(10) Pub. No.: US 2025/0285271 A1
(43) Pub. Date: Sep. 11, 2025

(54) IMAGING SYSTEM INCLUDING STORING MEDIUM HAVING TRAINED MODEL ENABLING RECONSTRUCTION OF MEDICAL IMAGE HAVING PREFERABLY FEATURE VALUE OF IMAGE QUALITY INDEX; AND METHOD FOR PRODUCING TRAINED MODEL

(71) Applicant: GE Precision Healthcare LLC, Waukesha, WI (US)

(72) Inventors: Yasuhiro Imai, Hino (JP); Risa Shigemasa, Hino (JP); Hisanori Tsunemine, Hino (JP)

(21) Appl. No.: 19/072,780

(22) Filed: Mar. 6, 2025

(30) Foreign Application Priority Data
Mar. 6, 2024 (JP) 2024-034034

(51) Int. Cl. G06T 7/00; G16H 30/40

(52) U.S. Cl. CPC G06T 7/0012; G16H 30/40; G06T 2207/10081; G06T 2207/10104; G06T 2207/10112; G06T 2207/20081; G06T 2207/20182; G06T 2207/30096

(57) ABSTRACT

A medical imaging described herein includes a model trained using a first medical image in which a first image quality index is prioritized and a second medical image in which a second image quality index is prioritized. Learning data includes a first algorithm and a first condition for processing data acquired to obtain the first medical image, a feature value of the first image quality index, a second algorithm and a second condition for processing data acquired to obtain the second medical image, and a feature value of the second image quality index. The model determines a first model algorithm and a first model parameter for obtaining a first model medical image in which the first image quality index is prioritized, and/or a second model algorithm and a second model parameter for obtaining a second model medical image in which the second image quality index is prioritized.

### CROSS-REFERENCE TO RELATED APPLICATIONS

This application claims priority to Japanese Application No. 2024-034034, filed on Mar. 6, 2024, the disclosure of which is incorporated herein by reference in its entirety.

### TECHNICAL FIELD

The present invention relates to a medical imaging system, and more particularly relates to a technology for analyzing a medical image using a trained model.

### BACKGROUND ART

When acquiring medical images, there is a demand for prioritizing a feature value of a specific type of image quality index over feature values of other types of image quality indexes depending on what is desired to be observed. For example, when observing hemorrhaging within the skull, such as epidural hematoma, subdural hematoma, subarachnoid hemorrhage, intracerebral hemorrhage, or the like, the difference in CT value between the intracranial tissue and the hemorrhaging portion is slight, and thus high density resolution and low noise level are required. On the other hand, hemorrhaging generally spreads over a certain range, and therefore, high spatial resolution is not required. Furthermore, for example, when observing a skull fracture (e.g., a fracture in which a linear crack has occurred), high density resolution is not required, and spatial resolution takes priority.

For example, to solve such problems when performing imaging using an X-ray CT device, a radiologist is required to set an appropriate acquisition parameter and reconstruction algorithm to perform imaging under various constraints, such as radiation dose restrictions and the like. There are a plurality of types of image quality indexes, such as spatial resolution, contrast resolution, noise, artifacts, and the like. Depending on the specific clinical purpose, certain types of image quality indexes are prioritized, and other image quality indexes must be maintained at a certain level, albeit relatively low. All image quality indexes cannot be improved, and improving one image quality index often leads to a decrease in another image quality index. Such settings are not necessarily easy for radiologists with low skill levels, and the settings require a lot of time and effort.

### SUMMARY OF THE INVENTION

Therefore, there is a need for an image generating system in which a preferable feature value of an image quality index can be easily specified. Furthermore, there is a need for a system in which a preferable feature value of an image quality index that is suited to the type and location of diagnosis can be easily and quickly identified, without requiring a high level of skill.

Furthermore, there is a need for a system that can easily obtain an image having the same image quality as an existing image.

In the present disclosure, a medical imaging system is provided, the system including a storing medium storing a trained model trained using, as learning data, a first medical image in which a first image quality index is prioritized and a second medical image in which a second image quality index, which is different from the first image quality index, is prioritized. The learning data includes, as annotations: at least one of a first algorithm and a first condition for processing data acquired to obtain the first medical image; a feature value of the first image quality index; at least one of a second algorithm and a second condition for processing data acquired to obtain the second medical image; and a feature value of the second image quality index. The trained model is configured to: determine first information containing at least one of a first model algorithm and a first model parameter for obtaining a first model medical image in which the first image quality index is prioritized, and a model feature value of the first image quality index; and determine second information containing at least one of a second model algorithm and a second model parameter for obtaining a second model medical image in which the second image quality index is prioritized, and a model feature value of the second image quality index. Furthermore, when medical image data and a selection of either the first image quality index or the second image quality index are input, the trained model processes the input medical image data using the first or second information according to input information on a user interface, and outputs a medical image having a model feature value of the first or second image quality index.

In another aspect of the present disclosure, a method is provided, the method being for producing a trained model trained using, as learning data, a first medical image in which a first image quality index is prioritized and a second medical image in which a second image quality index, which is different from the first image quality index, is prioritized. The method includes a step for generating learning data, the learning data including, as annotations: at least one of a first algorithm and a first condition for processing data acquired to obtain the first medical image; a feature value of the first image quality index; at least one of a second algorithm and a second condition for processing data acquired to obtain the second medical image; and a feature value of the second image quality index. The trained model determines at least one of a first model algorithm and a first model parameter for obtaining a first model medical image in which the first image quality index is prioritized, and determines at least one of a second model algorithm and a second model parameter for obtaining a second model medical image in which the second image quality index is prioritized.

### BRIEF DESCRIPTION OF THE DRAWINGS

FIG. 1 is a diagram schematically depicting a configuration of an X-ray CT system according to the present embodiment;

FIG. 2 is a diagram depicting a configuration of a main part of an X-ray tube and X-ray detecting part;

FIG. 3 is a diagram depicting learning stages for generating a trained model;

FIG. 4 is a diagram depicting a learning stage and inference stage using a trained model; and

FIG. 5 is a network diagram depicting a trained model.

### DETAILED DESCRIPTION OF THE DRAWINGS

Embodiments of the present invention will be described below. Note that the invention is not limited thereto.

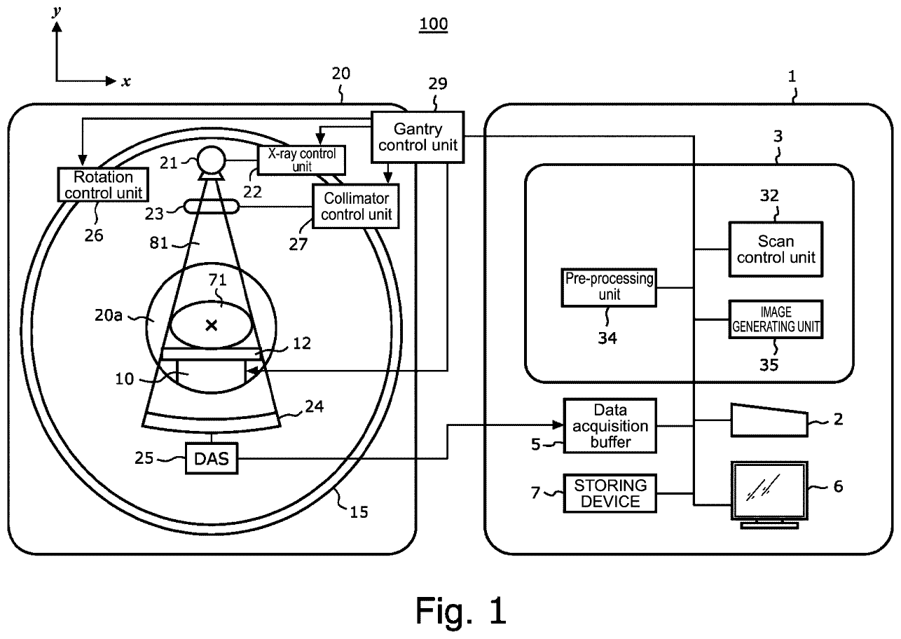

FIG. 1 is a block diagram depicting a configuration of an X-ray CT device 100 according to the present embodiment. In the present disclosure, a medical X-ray CT device is described as an example, but the present invention can be applied to non-destructive examining devices, such as dental CT devices, CT devices for inspecting baggage, and the like. The CT device can also be replaced with an MRI device, a PET device, a SPECT device, a tomosynthesis device, or the like. In the example of FIG. 1, the X-ray CT device 100 includes an operation console 1, an imaging table 10, and a scanning gantry 20. In a preferred embodiment of the present invention, a medical X-ray CT device that acquires projection data from a subject that is a human or a non-human animal and reconstructs an image will be described as an example, but the present invention can also be applied to a dental CT device, a CT device for inspecting baggage, a PET device, a SPECT device, a tomosynthesis device, and the like.

The operation console 1 has a configuration serving as a computer. Specifically, the operation console 1 includes: an input device 2, such as a keyboard, a mouse, or the like for receiving input from an operator; a central processing device 3 for executing scan control processing, pre-processing, image generation processing, and the like; and a data acquisition buffer 5 for acquiring X-ray detector data acquired by the scanning gantry 20. Furthermore, the operation console 1 includes: a monitor 6 for displaying a multi-energy image generated by the image generation processing; and a storing device 7 for storing a program, X-ray detector data, X-ray projection data, a dual-energy image, or the like. The imaging conditions are input from the input device 2 and stored in the storing device 7.

The imaging table 10 includes a cradle 12 on which a subject 71 is placed and which is moved in and out of an opening 20a (to be described later) of the scanning gantry 20. The cradle 12 is moved up and down and horizontally in a straight line by a motor internally provided in the imaging table 10.

The scanning gantry 20 has the opening 20a through which the subject 71 to be imaged is transported.

Furthermore, the scanning gantry 20 has an X-ray tube 21, an X-ray control unit 22 for controlling the X-ray tube voltage, X-ray irradiation timing, and the like in the X-ray tube 21, and a collimator 23 having an opening that shapes X-rays irradiated from the X-ray tube 21 into a fan-shaped X-ray beam 81. Furthermore, the scanning gantry 20 has: a collimator control unit 27 for controlling the opening of the collimator 23, an X-ray detector 24 for detecting X-rays irradiated from the X-ray tube 21, and a data acquisition system (DAS) 25 for acquiring X-ray detector data (also referred to as raw data) from an output of the X-ray detector 24. The DAS 25 samples analog data received from a detector element of the X-ray detector 24 and converts the analog data to digital signals for subsequent processing.

Furthermore, the scanning gantry 20 has: a gantry rotating part 15 that holds the X-ray tube 21, the collimator 23, and the X-ray detector 24 and rotates around the body axis of the subject 71; and a rotation control unit 26 that controls the gantry rotating unit 15. Furthermore, the scanning gantry 20 has a gantry control unit 29 for transmitting and receiving control signals between the operation console 1 and the X-ray control unit 22, rotation control unit 26, imaging table 10, and the like. Note that in implementation, the scanning gantry 20 includes a beam-forming X-ray filter that spatially controls the dose of the X-ray beam 81, and an X-ray filter that controls the radiation quality of the X-ray beam 81. The gantry rotating part 15 holds these filters between the collimator 23 and the opening 20a, and illustrations and detailed descriptions thereof are omitted herein.

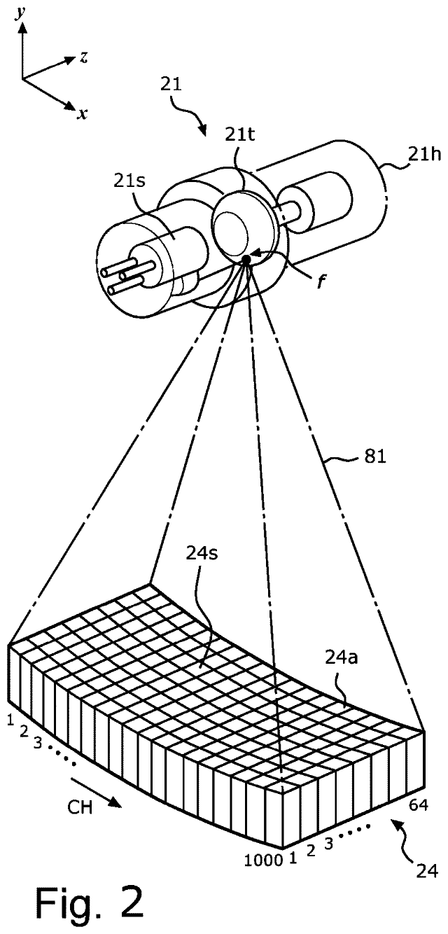

FIG. 2 is a diagram depicting a configuration of a main part of the X-ray tube 21 and X-ray detecting part 24. Herein, the vertical direction is defined as the y-axis direction, a transporting direction of the imaging table 10 (which usually coincides with a thickness direction of the X-ray beam 81 or the body axis direction of the subject 71) is defined as the z-axis direction, and the direction orthogonal to the y-axis and z-axis directions (channel direction) is defined as the x-axis direction.

These components are supported on a prescribed base part of the gantry rotating part 15 and maintain the positional relationship as depicted in the drawings. In other words, the X-ray tube 21 and the X-ray detector 24 are disposed opposite each other with the opening 20a therebetween. Furthermore, X-rays emitted from the X-ray tube 21 pass through a slit formed by the collimator 23 (not depicted in FIG. 2), forming a fan-shaped X-ray beam 81 having a prescribed thickness (cone angle) and spread (fan angle).

The X-ray tube 21 has a structure in which a cathode sleeve 21s incorporating a focusing electrode and a cathode filament, and a rotating target electrode 21t are housed in a housing 21h, and generates X-rays emitted from an X-ray focal point F.

The X-ray detecting part 24 is a so-called multi-row X-ray detector in which a plurality of detecting element rows, for example, 64 detecting element rows, are arranged in the z-axis direction (thickness direction of X-ray beam 81), each detecting element row being obtained by arranging a plurality of the X-ray detecting elements 24a, for example, 1,000 X-ray detecting elements in a channel direction CH (spread direction of X-ray beam 81). Herein, each of the detecting element rows are numbered 1, 2, 3, . . . , 64 from an end. This achieves a so-called 64-row multi-slice X-ray CT. However, the 64 detecting element rows herein are merely an example, and the present invention is not limited thereto. The X-ray detector 24 forms an X-ray detection surface 24s by the plurality of X-ray detecting elements 24a, the X-ray detection surface detecting the X-ray beam 81 that has passed through the subject 71. The X-ray detecting element 24a is configured as a so-called solid-state detector, for example, by combining a scintillator and a photodiode.

The central processing device 3 has a scan control unit 32, a pre-processing unit 34, and an image generating unit 35. The central processing device 3 is, for example, a processor such as a central processing unit (CPU) or the like. The central processing device 3 executes the functions of the scan control unit 32, pre-processing unit 34, and image generating unit 35 by reading and executing the program stored in the storing device 7. The program is an example of an embodiment of a control program according to the present invention.

The scan control unit 32 controls the X-ray control unit 22, the rotation control unit 26, the collimator control unit 27 and the imaging table 10 via the gantry control unit 29 so as to perform multi-energy imaging of the subject 71. Specifically, the scan control unit 32 controls the abovementioned units to rotate the X-ray tube 21 and the X-ray detector 24 around the subject 71 to acquire X-ray projection data.

In the present embodiment, the X-ray tube 21 can irradiate not only a monochromatic X-ray beam but also an arbitrary number of polychromatic X-ray beams equal to or greater than two. A tube voltage of an arbitrary value between 50 and 200 kV, such as 80 kV, 85 kV, 100 kV, 120 kV, 130 kV, 140 kV, 150 kV, 200 kV, or the like, is applied to the X-ray tube 21 by switching the voltage for each view to be acquired, and the X-ray tube 21 irradiates an X-ray beam having an energy spectrum corresponding to the applied tube voltage.

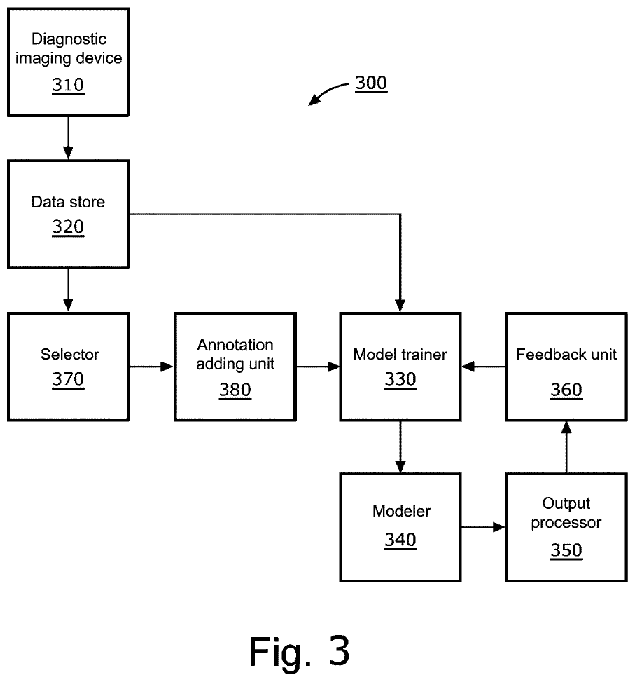

FIG. 3 depicts a trained model producing system 300. The trained model producing system 300 includes a diagnostic imaging device 310, a data store 320, a model trainer 330, a modeler 340, an output processor 350, a feedback unit 360, a selector 370, and an annotation adding unit 380. Each functional block can be executed by one or a plurality of processors. In the present embodiment, the diagnostic imaging device 310 in FIG. 3 corresponds to the X-ray CT device 100 in FIG. 1, and the data store 320 corresponds to the storing device 7 in FIG. 1. In another embodiment, the diagnostic imaging device 310 in FIG. 3 corresponds to a plurality of X-ray CT devices, including the X-ray CT device 100 in FIG. 1. In a specific example, the data store 320 is disposed in a database of a server to which the plurality of X-ray CT devices are connected via a network, and the data store 320, the model trainer 330, the modeler 340, the output processor 350, the feedback unit 360, the selector 370, and the annotation adding unit 380 are disposed in the central processing device 3 that functions as a trained model producing terminal. In another specific example, the data store 320, the model trainer 330, the modeler 340, the output processor 350, the feedback unit 360, the selector 370, and the annotation adding unit 380 are disposed in a server connected to the X-ray CT device 100 via the network. In yet another specific example, the data store 320, the model trainer 330, the modeler 340, the output processor 350, the feedback unit 360, the selector 370, and the annotation adding unit 380 are disposed in any one or more of the central processing devices 3 associated with a plurality of diagnostic imaging devices connected via the network. The data store 320, the model trainer 330, the modeler 340, the output processor 350, the feedback unit 360, the selector 370, and the annotation adding unit 380 can be processed by a plurality of processing devices distributed over a network. Image data to be analyzed is stored in the data store 320 (e.g., a database, a data structure, a hard drive, solid state memory, flash memory, other computer memory, or the like). The data store 320 may also be configured from a plurality of storing devices distributed across the network.

The data store 320 stores, in association with each other: one or more images acquired by the diagnostic imaging device 310 (such as the X-ray CT device 100 or the like in FIG. 1); an acquisition parameter used when acquiring the images; a reconstruction algorithm and an image reconstruction parameter used to reconstruct the images; and an examination objective. The acquisition parameter includes a tube voltage, a magnitude of a tube current, a slice width, a contrast agent protocol, and the like used in dual-energy imaging. If the examination objective is added by the annotation adding unit 380 (to be described later), the data store 320 does not store the examination objective. An image may be characterized by one or more of the following: spatial resolution, temporal resolution, contrast resolution, noise, and artifacts. There are various types of artifacts observed in CT images, such as metal objects, Poisson noise, Gaussian noise, streaks, scatter, and the like. Furthermore, these artifacts can be classified into types such as system design-induced artifacts (e.g., Gaussian noise and the like), X-ray tube-induced artifacts (e.g., focal point deviation, X-ray tube vibration, and the like), detector-induced artifacts (e.g., non-uniformity of detector response, and the like), patient-induced artifacts (e.g., patient movement during scan, metal objects, and the like), and operator-induced artifacts (e.g., insufficient exposure (low dose), improper slice width setting, and the like). Furthermore, low image quality may also be due to insufficient spatial resolution or insufficient contrast resolution.

The selector 370 receives a plurality of target images from the data store 320 and provides the function of selecting one or a plurality of specific cross-sections. For example, when the X-ray CT device 1 scans from the chest to the lower abdomen, several tens to several thousands of slice images can be generated depending on the slice width. The selector 370 provides various functions for selecting one or more radiological images from these images for adding an annotation. The selector 370 displays an image of the selected organ or site on the monitor 6 in response to the selection of the organ or site by an operator. The selected organ or site is automatically or manually enlarged as required. Furthermore, the selector 370 can have an image analyzing function. The selector 370 applies the trained AI model to the image being analyzed to identify the presence of a specific lesion, specific noise, or specific artifact in the image. The selector 370 can also use template matching or the like rather than an AI model to identify the presence of a particular lesion, particular noise, or particular artifact. Furthermore, the selector 370 may select a target image in accordance with the type of acquisition parameter, such as an image acquired with a monochromatic X-ray beam, an image acquired with a polychromatic X-ray beam, an image using a contrast agent, an image of the head (using a head-specific acquisition parameter), and the like. A training network 420 and a trained model 460 (to be described later) can be generated corresponding to the type of acquisition parameter.

In a preferred example of the present invention, the selector 370 can automatically extract an image from the data store 320 in order to facilitate annotation by the operator. The operator can also use a known image analyzing function, such as AI, pattern matching, and the like, provided in the selector 370 to extract an image in which a specific organ, a specific lesion, or a specific type of artifact is present, and display the image on the monitor 6.

In a preferred example of the present invention, if the operator selects a large number of images that are not suitable as training data, the selector 370 outputs a display and/or audio prompting the operator to further narrow down the images to be selected as training data.

When the selector 370 identifies an image to be used as training data in response to the operator, the operator can use the function of the annotation adding unit 380. In FIG. 3, the annotation adding unit 380 is a separate functional block from the selector 370, but the two can also be implemented as a single functional block.

By using deep learning technology, it has become possible to detect objects in images and classify images. Learning requires a large number of images and accompanying information (annotations). It is difficult to acquire and annotate the images needed for learning in an unbiased manner. The annotation adding unit 380 provides both automatic annotation and manual annotation functions, reducing the annotation work.

In a preferred embodiment of the present invention, the annotation adding unit 380 causes the image selected by the selector 370 to be displayed on the monitor 6. The operator can display the image in various forms according to preference, for example by enlarging or reducing the image, or the like. By using the function of the annotation adding unit 380, a first medical image is annotated with at least one of a first algorithm and a first condition for processing data acquired to obtain the first medical image, and a feature value of a first image quality index. Furthermore, a second medical image is annotated with at least one of a second algorithm and a second condition for processing data acquired to obtain the second medical image, and a feature value of a second image quality index.

For example, a medical image including hemorrhaging inside the skull is annotated with feature values of spatial resolution, density resolution, and noise level, as well as the type of image reconstruction algorithm that is important for achieving these feature values, the non-use of a specific image reconstruction algorithm, or the examination objective, to obtain training data. Furthermore, a medical image including a skull fracture is annotated with feature values of spatial resolution, density resolution, and noise level, as well as the type of image reconstruction algorithm that is important for achieving these feature values, the non-use of a specific image reconstruction algorithm, or the examination objective, to obtain training data.

The automatic and/or manual addition of annotations can be executed in various forms. In a specific embodiment of the present invention, the operator identifies the position of an organ and/or lesion by surrounding the desired position with a geometric shape such as a circle, ellipse, polygon including rectangles, a complex curve drawn using edge detection, and the like. The annotation adding unit 380 identifies the type of organ and/or lesion, and displays on the monitor 6 a list of examination objectives corresponding to the identified organ and/or lesion. For example, if a skull with a crack is identified through image recognition, examination objectives for skull fractures, cerebral hemorrhaging, and the like are listed. When the examination objective to be registered is displayed at the top of the list, the operator can simply press the enter key or a confirmation button to register (annotate) the examination objective. If the examination objective to be registered is displayed in a position other than the top of the list, the examination objective can be registered (annotated) by pressing the enter key or a confirmation button after selection.

Furthermore, the annotation adding unit 380 provides the operator with a function for manually adding an annotation. The annotation adding unit 380 provides the operator with a text box for inputting the examination objective and accepts an input from the operator. The operator can also adopt or modify the stored examination objective even if the examination objective is stored in the data store 320. Note that in addition to the type of lesion, such as hemorrhaging, tumors, and the like, the examination objective can include the examination site (skull, brain, lungs, abdomen, liver, and the like), age (lower doses are required for younger patients), sex, race, and attending physician (physician and physician B may have different preferences for image quality), and the like.

The trainer 330 and the modeler 340 receive a set of training data via the selector 370 and the annotation adding unit 380. When the data store 320 stores an examination objective, the trainer 330 and the modeler 340 can receive a set of training data from the data store 320 without going through the selector 370 and the annotation adding unit 380. The trainer 330 can identify the training network 420 in which training is performed according to the type of acquisition parameter, such as an image acquired with a monochromatic X-ray beam, an image acquired with a polychromatic X-ray beam, an image using a contrast agent, an image of the head (using a head-specific acquisition parameter), and the like.

In a preferred embodiment of the present application, the trainer 330 iteratively trains the training network 420 to take an examination objective and image as inputs and to output a reconstruction algorithm and reconstruction parameter. During training, a first medical image, in which a first image quality index is prioritized, and a second medical image, in which a second image quality index different from the first image quality index is prioritized, are used as learning data. For example, in a medical image that includes hemorrhaging within the skull, such as epidural hematoma, subdural hematoma, subarachnoid hemorrhage, intracerebral hemorrhage, or the like, high density resolution and a low noise level are prioritized over spatial resolution, and such an image is used as learning data that prioritizes density resolution/noise level. An image that includes a skull fracture (e.g., a fracture with a linear crack) is prioritized in spatial resolution over density resolution, and such an image is also used as learning data with spatial resolution prioritized.

In another embodiment of the present application, the trainer 330 iteratively trains the training network 420 to take an examination objective and image quality index feature value as inputs and to output a reconstruction algorithm and reconstruction parameter. Input of a feature value of an image quality index extracted from an image into the network rather than the image itself can be achieved by internally or externally providing the trainer 330 with an image analyzing function for analyzing the image and outputting an image quality index feature value. When analyzing an image, the spatial resolution, contrast resolution, temporal resolution, noise, and artifact of the image are analyzed, and the feature values of such items are analyzed. The feature value can be specified by a noise value, a noise power spectrum (NPS), a modulated transfer function (MTF), or the like.

The modeler 340 uses a deployed artificial intelligence model (a trained model, e.g., neural network, random forest, or the like) to determine, in response to an input indicating a preference for the first image quality index, at least one of a first model algorithm and first model parameter for obtaining a first model medical image in which the first image quality index is prioritized, and to determine, in response to an input indicating a preference for the second image quality index, at least one of a second model algorithm and second model parameter for obtaining a second model medical image in which the second image quality index is prioritized.

The modeler 340 can use the deployed artificial intelligence model to receive the examination objective and image quality index feature value, which are input from the input device 2, and identify a reconstruction algorithm and/or reconstruction parameter to use. Furthermore, in response to a sample medical image being input, the modeler 340 can identify features of the first and/or second image quality index in the sample medical image, and output a reconstruction algorithm and/or reconstruction parameter for outputting a medical image having model feature values of the first and/or second image quality index. Furthermore, in response to medical image data and a selection to prioritize either the first image quality index or the second image quality index being input, the modeler 340 processes the input medical image data and outputs a reconstruction algorithm and/or reconstruction parameter for outputting a medical image having model feature values of the prioritized first or second image quality index. In a specific example, in response to an examination objective being input, the modeler 340 can identify a model feature value of an image quality index that matches the examination objective and output a reconstruction algorithm and/or reconstruction parameter for outputting a medical image having the model feature value. In other words, a model feature value of the image quality index is indirectly selected by identifying the examination objective. Identification of a model feature value of an image quality index that matches the examination objective can be performed for each attending physician, for each hospital, or for each affiliated hospital group having a plurality of hospitals. In a specific embodiment, identification of a model feature value of an image quality index that matches an examination objective can be customized by re-learning of the trained model 460. Although performing re-learning for each attending physician can provide an image quality that meets the needs of the attending physician, there is a possibility that the amount of learning data may be insufficient. Conversely, performing re-learning for each affiliated hospital group having a plurality of hospitals may increase the possibility of obtaining a sufficient amount of learning data, but may decrease the possibility of simultaneously satisfying the different preferences of individual medical image interpreters. The deployed trained model 460 can also be shared between affiliated hospitals. When systems with different image reconstruction capabilities are introduced between affiliated hospitals (e.g., in a certain image reconstruction algorithm, a higher-level model can set an image reconstruction parameter in the range of 1 to 10, whereas a lower-level model can only set the image reconstruction parameter in the range of 1 to 5, or the like), a trained model 460 for a higher-level model and a trained model 460 for a lower-level model may be provided. Furthermore, the trained model 460 can undergo continuous learning at each of the affiliated hospitals. Data captured on a different type of system than the system that provided an image to the trainer 330 may also be used for continuous learning. In this case, the trained model 460 can be customized. Conversely, if continuous learning is implemented across affiliated hospitals, the amount of learning data can be increased.

The reconstruction algorithm includes analytical image reconstruction methods, filtered back projection methods, adaptive iterative reconstruction methods, iterative reconstruction methods, model-based iterative reconstruction methods, deep learning image reconstruction methods, and other image reconstruction methods, as well as algorithms used in the image reconstruction methods. The algorithms used in the image reconstruction methods include algorithms for removing various artifacts, algorithms for removing noise, and the like. The reconstruction parameter includes parameters used in the image reconstruction methods, flags specifying ON/OFF selection of algorithms used in the image reconstruction methods, and parameters used in the algorithms used in each image reconstruction method.

In a preferred embodiment of the present application, a simulated image that is expected to be obtained when a specified reconstruction algorithm and/or reconstruction parameter is applied is displayed on the monitor 6 together with the reconstruction algorithm and the reconstruction parameter. Furthermore, the monitor 6 displays a numerical value corresponding to a feature value of an image quality index (including spatial resolution, contrast resolution, noise, artifacts, and the like) of the simulated image. An operator and/or physician of the X-ray CT device 1 determines the validity of a re-acquisition algorithm and reconstruction parameter generated by the modeler 340, and if valid, the image generating unit 35 performs image reconstruction using the re-acquisition algorithm and reconstruction parameter generated by the modeler 340. The determination of validity may occur before or after the acquisition of the projection data to be reconstructed.

If the simulated image is not valid, the operator can modify the numerical value corresponding to the feature value of the image quality index (including spatial resolution, contrast resolution, noise, artifacts, and the like) of the simulated image displayed on the monitor 6 via the input device 2. The modeler 340 again outputs the reconstruction algorithm and reconstruction parameter on the basis of the feature value of the modified image quality index. Furthermore, a new simulated image, reconstruction algorithm and/or reconstruction parameter and image quality index feature value corresponding to the modified numerical value are displayed on the monitor 6. The reconstruction algorithm and/or reconstruction parameter generated by modeler 340 are modified accordingly. If the determination of validity occurs prior to acquisition of the projection data to be reconstructed, an acquisition parameter may be modified as necessary to obtain the ideal image quality.

If the operator determines that the image reconstructed using the re-acquisition algorithm and reconstruction parameter generated by the modeler 340 that were deemed to be valid is in fact invalid, the operator can modify the numerical value corresponding to the feature value of the image quality index (including spatial resolution, contrast resolution, noise, artifacts, and the like) of the image displayed on the monitor 6 via the input device 2. A new simulated image, reconstruction algorithm and/or reconstruction parameter and image quality index feature value corresponding to the modified numerical value are displayed on the monitor 6. The reconstruction algorithm and/or reconstruction parameter generated by modeler 340 are modified accordingly. If the output of the trained model 460 is inappropriate, such as when an image reconstructed using the re-acquisition algorithm and reconstruction parameter generated by the modeler 340 that were deemed valid significantly differs from the original simulated image, or the like, the result thereof can be delivered to the feedback unit 360 (to be described later), and retraining of the training network 420 can be performed.

If feedback from an operator is provided to the trainer 330 via the feedback unit 360, the trainer 330 can use the feedback to improve the artificial intelligence model. For example, the trainer 330 may adjust (lower) the weighting of a node and change a connection between nodes in a learning model network used to obtain the output that caused the feedback. Conversely, if no feedback is provided and the output is accepted by the user, the weighting of the node in the learning model network used for the output can be strengthened.

In a preferred example of the present invention, the trained model 460 is transmitted to: a central managing device for managing a plurality of X-ray CT devices or other imaging devices; and/or another imaging device. In the X-ray CT device 1 and/or other imaging device, automatic and/or manual re-learning of the trained model 460 is executed.

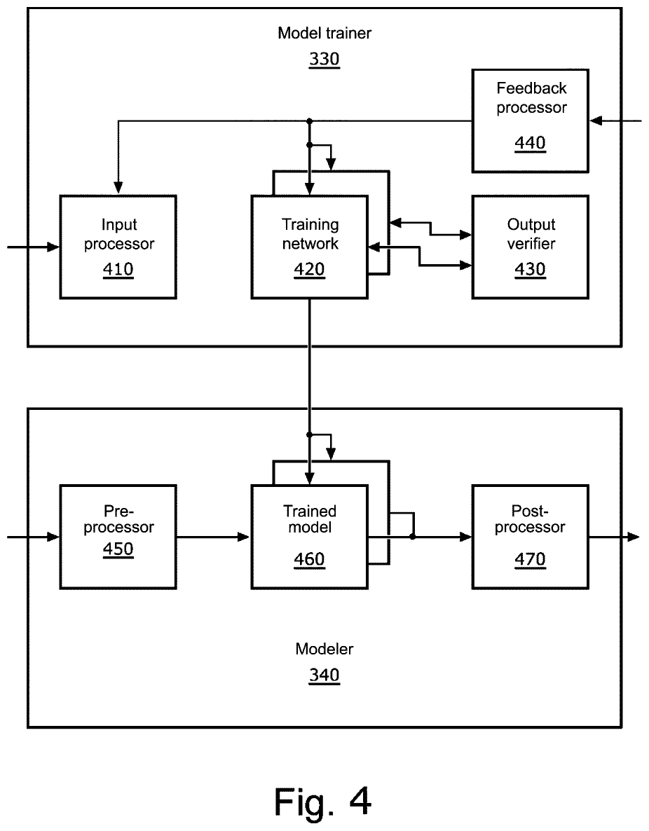

FIG. 4 depicts an exemplary embodiment of the trainer 330 and the modeler 340. As depicted in the example of FIG. 4, the trainer 330 includes an input processor 410, a training network 420, an output verifier 430, and a feedback processor 440. In this example, the modeler 340 includes a pre-processor 450, a trained model 460, which is a deployed model, and a post-processor 470. In the example of FIG. 4, an input such as an image, acquisition parameter and/or reconstruction parameter, annotation, or the like received directly from the data store 320 or via the selector 370 are provided to the input processor 410, which selects a training network 420 from among a plurality of training networks 420 corresponding to the input and prepares data to be input to the training network 420. For example, the data may be changed in such a manner where the image is filtered, supplemented, and/or with unnecessary portions removed, and the like, such that information input to the network 420 is more suitable for learning.

The training network 420 of the example of the drawing analyzes input data from the input processor 410 and generates an output that is verified by the output verifier 430. The output verifier 430 may, for example, verify the accuracy of the output from the training network 420. If the reconstruction algorithm or reconstruction parameter is outside the usable range, or the like, the output is deemed to be incorrect. If the output of the network 420 is not accurate, then, for example, the network 420 can be updated and modified (e.g., by adjusting network weighting, changing a node connection, or the like) to generate a correct output. Once the output of the training network 420 has been validated by the output verifier 430, the training network 420 can be used to generate and deploy the trained model 460 for the modeler 340.

Feedback can be periodically input to the feedback processor 440, which processes the feedback and evaluates whether to trigger an update or regeneration of the network 420. For example, if the output of the deployed trained model 460 continues to be accurate, there may be no reason to perform an update. However, for example, if the output of trained model 460 becomes inaccurate, this may trigger a regeneration or other update of the network 420 and an updated trained model 460 may be deployed (e.g., on the basis of additional data, a new constraint, an updated configuration, or the like).

The deployed trained model 460 is used by the modeler 340 to process the input image data and identify a reconstruction algorithm and reconstruction parameter. Input data from the data store 320 or the like is prepared by the pre-processor 450, and the input data is provided to the trained model 460 (e.g., a deep learning network model, a machine learning model, another network model, or the like) selected by pre-processor 450 in response to the input image data. The pre-processor 450 can adjust the input image data, such as adjusting the decimation (e.g., removing even slices, and the like), contrast, brightness, levels, artifacts/noise, or the like, before the image data is provided to the trained model 460. In a specific embodiment, a medical image is input to the trained model 460. In another embodiment, a feature value of an image quality index extracted from a medical image is input to the trained model 460 rather than the medical image. When the feature values of image quality indexes are input to the trained model 460, the pre-processor 450 analyzes the input medical image, identifies the feature values for each analyzed image quality index, and delivers the feature values to the trained model 460.

The trained model 460 processes the data from the pre-processor 450 and outputs a reconstruction algorithm and reconstruction parameter. The output from the trained model 460 is post-processed by the post-processor 470, which can clean up, organize, and/or otherwise modify the output data to form a composite 2D image. In a specific example, the post-processor 470 can generate a simulated image corresponding to the output reconstruction algorithm and reconstruction parameter, and can verify and/or otherwise execute a quality check on the output simulated image, reconstruction algorithm, and/or reconstruction parameter before the generated simulated image is stored, displayed, transferred to another system, or the like.

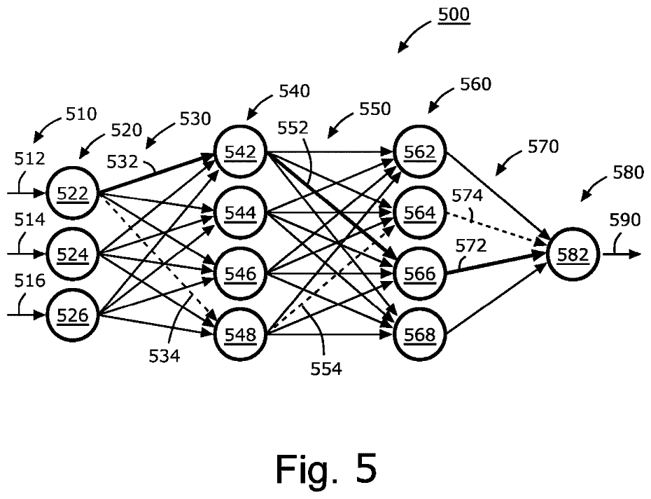

FIG. 5 depicts an exemplary deep learning neural network 500. The exemplary neural network 500 includes layers 520, 540, 560, and 580. The layers 520 and 540 are connected by a neural connection 530. The layers 540 and 560 are connected by a neural connection 550. The layers 560 and 580 are connected by a neural connection 570. Data flows forward from the input layer 520 via inputs 512, 514, and 516 to the output layer 580 and an output 590.

The layer 520 is an input layer that includes a plurality of nodes 522, 524, and 526 in the example of FIG. 5. The layers 540 and 560 are hidden layers and include nodes 542, 544, 546, 548, 562, 564, 566, and 568 in the example of FIG. 5. The neural network 500 may include more or fewer hidden layers 540 and 560 than are depicted. The layer 580 is an output layer and, includes a node 582 having the output 590 in the example of FIG. 5. Each of the inputs 512 to 516 corresponds to the nodes 522 to 526 in the input layer 520, and each of the nodes 522 to 526 in the input layer 520 has the connection 530 to the respective nodes 542 to 548 in the hidden layer 540. Each of the nodes 542 to 548 in hidden layer 540 has the connection 550 to the respective nodes 562 to 568 in the hidden layer 560. Each of the nodes 562 to 568 in the hidden layer 560 has the connection 570 to the output layer 580. The output layer 580 has the output 590, which provides an output from the exemplary neural network 500.

Of the connections 530, 550, and 570, specific exemplary connections 532, 552, and 572 may be provided with additional weighting, while other exemplary connections 534, 554, and 574 may be provided with a lower weighting in the neural network 500. The input nodes 522 to 526 are activated, for example, by receiving input data via the inputs 512 to 516. The nodes 542 to 548 and 562 to 568 in the hidden layers 540 and 560 are activated by the forward flow of data in the network 500 via the connections 530 and 550, respectively. The node 582 in the output layer 580 is activated after the data processed in the hidden layers 540 and 560 is sent via the connection 570. When the output node 582 in the output layer 580 is activated, the node 582 outputs an appropriate value on the basis of the processing accomplished in the hidden layers 540 and 560 of the neural network 500. Images, noise and artifacts that appear in specific points in the image, and positional information thereof can be used to create models used for object detection, shape detection (segmentation), and classification learning. Furthermore, a model can be created on the basis of frequency analysis and the like for the spatial resolution, contrast resolution, and temporal resolution of an image.

The present invention has been described above with a focus on the most preferred embodiment. However, as will be apparent to a person of ordinary skill in the art, the present invention can be implemented by making various changes and modifications to the embodiments within the technical scope of the present invention.

Furthermore, a program for causing a computer to function as each means for controlling and processing the X-ray CT device is also an example of an embodiment of the invention.

1. A medical imaging system, comprising a storing medium storing a trained model trained using, as learning data, a first medical image in which a first image quality index is prioritized and a second medical image in which a second image quality index, which is different from the first image quality index, is prioritized, wherein
the learning data includes,
as annotations, at least one of a first algorithm and a first condition for processing data acquired to obtain the first medical image, a feature value of the first image quality index, wherein at least one of a second algorithm and a second condition for processing data acquired to obtain the second medical image; and a feature value of the second image quality index; and
the trained model is configured to:
determine at least one of a first model algorithm and a first model parameter for obtaining a first model medical image in which the first image quality index is prioritized; and
determine at least one of a second model algorithm and a second model parameter for obtaining a second model medical image in which the second image quality index is prioritized.

2. The medical imaging system according to claim 1, wherein
the trained model processes,
in response to medical image data and a selection of either the first image quality index or the second image quality index being directly or indirectly input, the input medical image data and then outputs a reconstruction algorithm and/or reconstruction parameter for outputting a medical image having a model feature value of the first or second image quality index, and
the first image quality index and/or the second image quality index is indirectly selected by selecting an examination objective.

3. The medical imaging system according to claim 1, wherein the first image quality index is one or more of spatial resolution, contrast resolution, temporal resolution, noise, and an artifact,
the second image quality index is one or more of spatial resolution, contrast resolution, temporal resolution, noise, and an artifact, and
the feature value of the first and/or second image quality index includes any one of a noise value, a noise power spectrum (NPS), and a modulated transfer function (MTF).

4. The medical imaging system according to claim 3, wherein the first and second medical images are reconstructed on the basis of a projection signal acquired by a radiation imaging device.

5. The medical imaging system according to claim 4, wherein the radiation imaging device is any one of a CT device, a PET device, a SPECT device, and a tomosynthesis device.

6. The medical imaging system according to claim 5, wherein the projection signal is a signal acquired from a subject, the subject being a human or a non-human animal; and
the first and/or second conditions include information on an examination objective of the subject, a lesion present or suspected to be present in the subject, and/or a specific site on the subject.

7. The medical imaging system according to claim 6, wherein the trained model includes one or more trained models associated with one or a plurality of acquisition parameters used when the projection signal is acquired, the examination objective, the lesion and/or the site.

8. The medical imaging system according to claim 1, wherein the trained model is configured to select a reconstruction algorithm to which the reconstruction parameter is applied from among a plurality of reconstruction algorithms on the basis of the first and/or second conditions;
the second model parameter includes a flag indicating non-use of the first model algorithm; and
the selected reconstruction algorithm is one or more of an analytical image reconstruction method, a filtered back projection method, an adaptive iterative reconstruction method, an iterative reconstruction method, a model-based iterative reconstruction method, a deep learning image reconstruction method, and an artifact removal algorithm.

9. The medical imaging system according to claim 8, further comprising a user interface including an inputting device for accepting an operator input and a display device for displaying the reconstructed image, wherein
the input device is configured to accept input of the first image quality index and/or the second image quality index,
the display device is configured to display a numerical value corresponding to a feature value of an image quality index of a reconstructed image currently displayed on the display device,
an operator can modify the numerical value via the input device, and
the display device is further configured to display a reconstructed image having a feature value of the image quality index corresponding to the modified numerical value.

10. A method for producing a trained model trained using, as learning data, a first medical image in which a first image quality index is prioritized and a second medical image in which a second image quality index, which is different from the first image quality index, is prioritized, the method comprising
a step for generating learning data, the learning data including,
as annotations: at least one of a first algorithm and a first condition for processing data acquired to obtain the first medical image; a feature value of the first image quality index; at least one of a second algorithm and a second condition for processing data acquired to obtain the second medical image; and a feature value of the second image quality index, wherein
the trained model is configured to:
determine at least one of a first model algorithm and a first model parameter for obtaining a first model medical image in which the first image quality index is prioritized; and
determine at least one of a second model algorithm and a second model parameter for obtaining a second model medical image in which the second image quality index is prioritized.

11. The method according to claim 10, wherein
the trained model processes,
in response to medical image data and a selection of either the first image quality index or the second image quality index being directly or indirectly input, the input medical image data and then outputs a reconstruction algorithm and/or reconstruction parameter for outputting a medical image having a model feature value of the first or second image quality index, and
the first image quality index and/or the second image quality index is indirectly selected by selecting an examination objective.

12. The method according to claim 10, wherein the first image quality index is one or more of spatial resolution, contrast resolution, temporal resolution, noise, and an artifact,
the second image quality index is one or more of spatial resolution, contrast resolution, temporal resolution, noise, and an artifact, and
the feature value of the first and/or second image quality index includes any one of a noise value, a noise power spectrum (NPS), and a modulated transfer function (MTF).

13. The method according to claim 12, wherein the first and second medical images are reconstructed on the basis of a projection signal acquired by a radiation imaging device.

14. The method according to claim 13, wherein the radiation imaging device is any one of a CT device, a PET device, a SPECT device, and a tomosynthesis device.

15. The method according to claim 14, wherein the projection signal is a signal acquired from a subject, the subject being a human or a non-human animal; and
the first and/or second conditions include information on an examination objective of the subject, a lesion present or suspected to be present in the subject, and/or a specific site on the subject.

16. The method according to claim 15, wherein the trained model includes one or more trained models associated with one or a plurality of acquisition parameters used when the projection signal is acquired, the examination objective, the lesion and/or the site.

17. The method according to claim 10, wherein the trained model is configured to select a reconstruction algorithm to which the reconstruction parameter is applied from among a plurality of reconstruction algorithms on the basis of the first and/or second conditions,
the second model parameter includes a flag indicating non-use of the first model algorithm, and
the selected reconstruction algorithm is one or more of an analytical image reconstruction method, a filtered back projection method, an adaptive iterative reconstruction method, an iterative reconstruction method, a model-based iterative reconstruction method, a deep learning image reconstruction method, and an artifact removal algorithm.

18. The method according to claim 17, further comprising:
a step for accepting, from an input device, an input of the first image quality index and/or the second image quality index; and
a step for displaying a numerical value corresponding to a feature value of an image quality index of a reconstructed image currently displayed on a display device, wherein
the numerical value is modifiable via the input device, and
the display device is further configured to display a reconstructed image having a feature value of the image quality index corresponding to the modified numerical value.
